(19) United States
(12) Patent Application Publication
(10) Pub. No.: US 2025/0290926 A1
(43) Pub. Date: Sep. 18, 2025

(54) UNDER-OIL EXTRACTION USING EXCLUSIVE LIQUID REPELLENCY FOR CELL ISOLATION

(71) Applicant: Wisconsin Alumni Research Foundation, Madison, WI (US)

(72) Inventors: David J. Beebe, Monona, WI (US); Terry D. Juang, Madison, WI (US); Sheena C. Kerr, Waunakee, WI (US)

(21) Appl. No.: 18/606,676

(22) Filed: Mar. 15, 2024

(51) Int. Cl. G01N 33/569; B01L 3/00; G01N 33/543

(52) U.S. Cl. CPC G01N 33/56972; B01L 3/502761; G01N 33/54326; B01L 2200/0652; B01L 2200/0673; B01L 2300/0645; B01L 2300/0663

(57) ABSTRACT

Disclosed herein are methods, systems, and kits for separating a cell in an aqueous sample, cell comprising a primary binding member, a magnetic particle comprising a particle binding site, a secondary binding member comprising a primary binding member binding site reactive to the primary binding member, and a binding site reactive to the particle binding site. The methods and systems expose the aqueous sample to the secondary binding member and to the magnetic particle to form a selected mixture including: a magnetically tagged cell of the secondary binding member bound to the particle binding site of the magnetic particle, and the secondary binding member bound to the primary binding member of the cell. Further, the selected mixture is placed on a surface of a substrate having an oil and is exposed to a magnetic field, magnetically attracting the magnetically tagged cell to the magnetic field, thereby separating the magnetically tagged cell from the aqueous sample to produce a separated aqueous sample.

### STATEMENT REGARDING FEDERALLY SPONSORED RESEARCH

This invention was made with government support under AI152177, AI154940, and CA247479 awarded by the National Institutes of Health. The government has certain rights in the invention.

### CROSS-REFERENCE TO RELATED APPLICATIONS

N/A

### FIELD OF THE INVENTION

The disclosed technology is generally directed to cell isolation and separation. More particularly the technology is directed to under-oil cell isolation and separation.

### BACKGROUND OF THE INVENTION

Magnetic cell separation is a common method for isolating immune cells from blood, which utilizes magnetic particles coated with antibodies to selectively isolate target immune cells from a mixture. However, often necessitates large sample volumes, multiple isolation steps, and extended isolation time.

Accordingly, there is a need for methods, systems, and kits to enable small sample volume, fast, and low sample loss cell isolations.

### BRIEF SUMMARY OF THE INVENTION

In one embodiment the disclosure provides a method for cell separation, including: providing an aqueous sample including a first cell, the first cell including a first primary binding member; providing a magnetic particle including a particle binding site; providing a first secondary binding member including a first primary binding member binding site reactive to the first primary binding member and a binding site reactive to the particle binding site; exposing the aqueous sample to the first secondary binding member and to the magnetic particle to form a selected mixture including: a first magnetically tagged cell including: the first secondary binding member bound to the particle binding site of the magnetic particle, and the first secondary binding member bound to the first primary binding member of the first cell; placing the selected mixture on a surface of a substrate, the surface including an oil; exposing the selected mixture to a magnetic field and magnetically attracting the first magnetically tagged cell to the magnetic field; and separating the first magnetically tagged cell from the aqueous sample to produce a separated aqueous sample.

In another embodiment the disclosure provides a system for cell separation, including: a secondary binding member including a primary binding member binding site reactive to a primary binding member and a binding site reactive to a magnetic particle binding site; a magnetic particle including a magnetic particle binding site corresponding to the secondary binding member; a surface of a substrate, the surface including an oil; a source of a magnetic field; and a set of instructions executed by a liquid handling machine including: exposing an aqueous sample, including a cell including a primary binding member to the magnetic particle and to the secondary binding member to form a selected mixture including: a magnetically tagged cell including: the secondary binding member bound to the magnetic particle binding site of the magnetic particle, and the secondary binding member bound to the primary binding member of the cell; placing the selected mixture on a surface of a substrate, the surface including an oil; exposing the selected mixture to a magnetic field and magnetically attracting the magnetically tagged cell to the magnetic field; and separating the magnetically tagged cell from the aqueous sample to produce a separated aqueous sample substantially free of the magnetically tagged cell.

In yet another embodiment the disclosure provides a kit for cell separation, including: a secondary binding member including a primary binding member binding site reactive to a primary binding member and a binding site reactive to a magnetic particle binding site; a magnetic particle including a magnetic particle binding site corresponding to the antibody; a surface of a substrate, the surface including an oil; and a source of a magnetic field.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows a schematic illustration of some embodiments of the methods disclosed herein.

FIG. 2 shows a schematic illustration of some embodiments of the systems disclosed herein.

FIG. 3 shows an embodiment of the methods disclosed herein. To isolate immune cells, inventors mix whole blood with special cocktails and magnetic beads. The mixture is placed into a vial filled with silicone oil (FIG. 3A). After an incubation time, Inventors then use a pipette with magnets to pull out unwanted cells from the top, leaving the immune cells of interest at the bottom droplet (FIG. 3B). FIG. 3C shows a photograph of a blood droplet before Inventors removed the unwanted cells, and FIG. 3D shows a photograph after, with the immune cells remaining at the bottom of the vial.

FIG. 4 shows fluorescence microscopy analysis of cells purified by the methods disclosed herein. Peripheral blood mononuclear cells (neutrophils, monocytes, T cells, and natural killer (NK) cells), were isolated from microvolume blood samples (8 μl) obtained from a single donor. The non-target cells were removed with a magnet for 1 min, leaving the purified, target cells to be collected by pipetting. The purified cells were stained with Hoechst dye for both quantification and morphological assessment. Analysis of the stained cells revealed that the frequency of isolated cells is consistent with the known frequencies of these cells in whole blood, indicating that the method disclosed herein provide a uniform isolation efficiency across different cell types.

FIG. 5 shows purity and gene expression analyses of T-cells purified by the methods disclosed herein. FIG. 5A shows a fluorescence microscopy image of isolated T cells stained with Hoechst at 10 μg/ml to visualize nuclei and with an anti-CD3 antibody to confirm T cell purity. Cells were quantified based on the presence of CD3 stained nuclei compared to non CD3 positive nuclei resulting in an approximate purity of 95%. FIG. 5B shows gene expression of T cells isolated by the methods disclosed herein (ELR-labeled bars) or by standard volume methods (STD-labeled bars). Post-isolation, T cells were either stimulated with ImmunoCult™ Human CD3/CD28 T Cell Activator (CD3/28 labeled bars) or left unstimulated as a control (CTRL-labeled bars). Gene expression analyses for T-helper type 1 (Th1, represented by IFN gamma) and type 2 (Th2, represented by IL-4 and IL-13) associated genes were performed using quantitative PCR (qPCR). Gene expression data were compared and normalized against three house-keeping genes. These genes were consistent across multiple donors and highly similar between the methods disclosed herein and standard isolation methods, indicating equivalent functionality between T cells isolated by and those isolated using standard volume (STD) methods. FIG. 5C shows IFN-gamma, IL-4 and IL-13 cytokine expression of isolated T cells (ELRi-labeled plots) or by standard volume methods (benchmark-labeled plots), as a measure of Th1 and Th2 responses. Cells were treated with ImmunoCult™ Human CD3/CD28 T Cell Activator (CD3/CD28, grey bars) or an unstimulated control (Control, black bars). These results corroborate the findings of FIG. 5B, with similar cytokine secretion patterns observed in both cells isolated by the methods disclosed herein and standard preparations across three different donors (D1, D2, D3).

### DETAILED DESCRIPTION OF THE INVENTION

Disclosed herein are methods, systems, and kits to enable small sample volume, fast, and low sample loss cell isolations. The methods, systems, and kits disclosed herein enable cell isolation, cell separation, cell purification, and/or cell concentration.

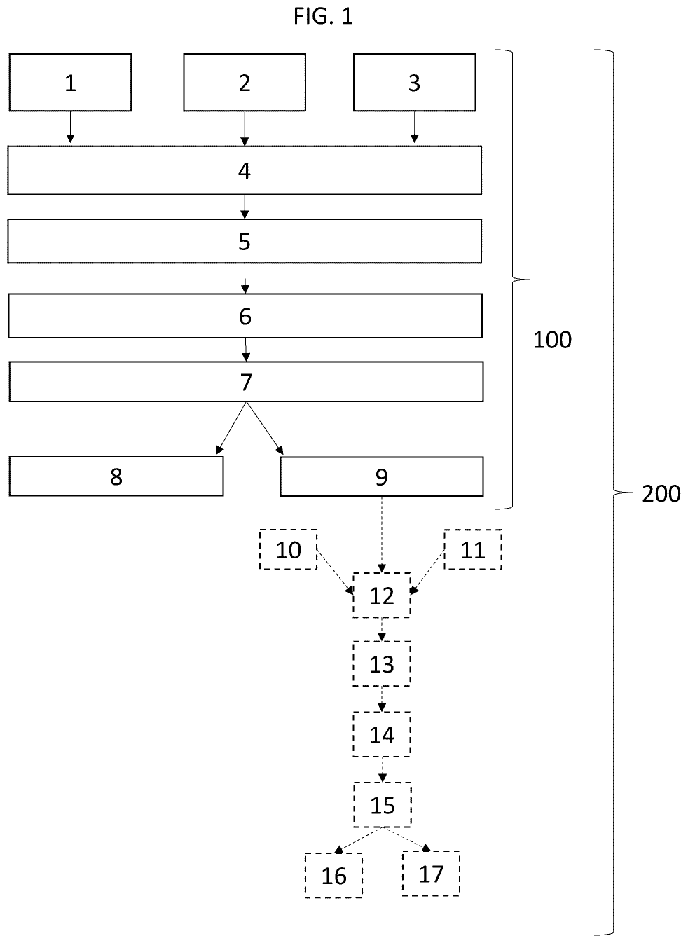

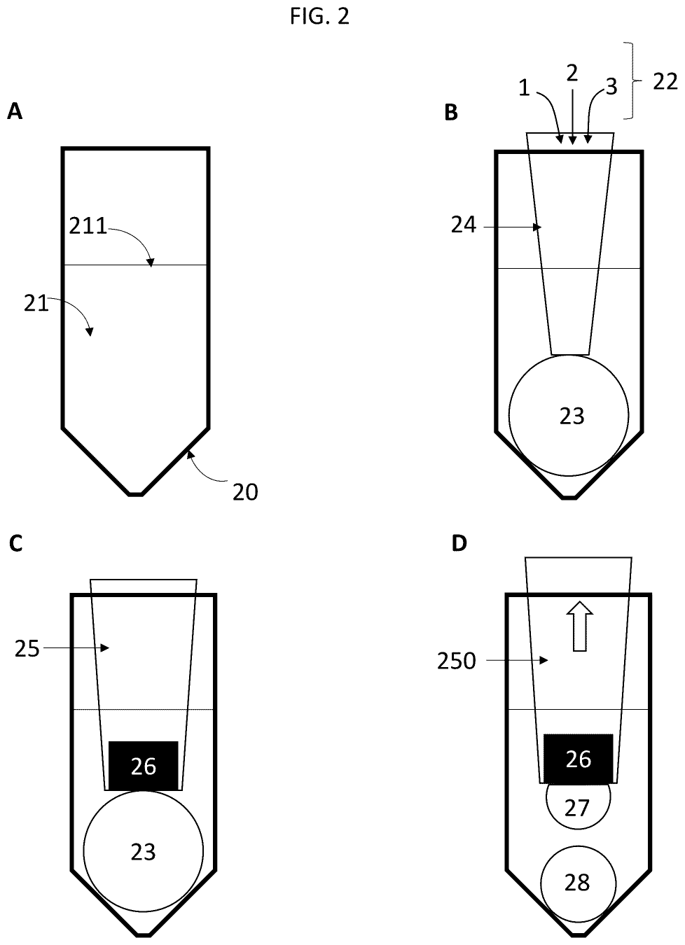

Referring to FIG. 1, disclosed herein is a method for cell separation including providing an aqueous sample (1) including a first cell, the first cell including a first primary binding member, providing a magnetic particle (2) including a particle binding site, and providing a first secondary binding member (3) including a first primary binding member binding site reactive to the first primary binding member and a binding site reactive to the particle binding site. The method includes exposing the aqueous sample to the first secondary binding member and to the magnetic particle to form a selected mixture (4) including a first magnetically tagged cell with: the first secondary binding member bound to the particle binding site of the magnetic particle, and the first secondary binding member bound to the first primary binding member of the first cell. The method includes placing the selected mixture on a surface of a substrate (5), the surface including an oil, exposing the selected mixture to a magnetic field (6) and magnetically attracting the first magnetically tagged cell to the magnetic field, and separating (7) the first magnetically tagged cell (8) from the aqueous sample to produce a separated aqueous sample (9).

The systems, methods, kits disclosed herein involve the concept of undermedia perfect liquid repellency. As used herein, “undermedia perfect liquid repellency” or, more specifically “underoil perfect liquid repellency,” refers to a state of matter whereby a three-phase system including a surface of a substrate, a dispersed phase of aqueous selected mixture and a continuous phase of oil give rise to complete repellency of the droplet of dispersed phase of aqueous selected mixture by the continuous phase of oil about the surface. It has been observed that this underoil perfect liquid repellency is attainable under the right conditions. A more thorough explanation of underoil perfect liquid repellency and the conditions under which it can be attained can be found in U.S. Patent Application Publication No. 2021/0138451 A1, which is incorporated by reference in its entirety and for all purposes herein.

In brief, for a surface to stably exist, its surface tension must be greater than zero. Otherwise, the atoms constituting a solid or a liquid will quickly diffuse into air. However, for an interface formed by two non-gas phases (e.g., a S/L interface) the S/L interfacial tension can be either positive, negative or zero and is directly determined by the interaction between the two phases. If the S/L interfacial tension is set equal to the sum of the surface tensions of each component phase, the interaction between the two phases is negligible (or zero) compared with the interaction from each bulk. In this case, the two phases are completely separated, which makes the interface between the two phases disappear. In Young's equation γ_{S/L}=γ_{S/G}−γ_{L/G} cos θ (where γ_{S/G} is solid surface tension, γ_{L/G} is liquid surface tension, γ_{S/L} is S/L interfacial tension, and θ is the inherent contact angle) and if θ is set as 180°, then γ_{S/L}=γ_{S/G}+γ_{L/G} can be derived yet is obviously at odds with the thermodynamic boundary condition of S/L interface (γ_{S/L}<γ_{S/G}+γ_{L/G}). This is consistent with empirical observations that no solid can perfectly repel liquid in air with long-term stability. Here it's interesting to see that if γ_{S/G} is set to 0 and γ_{S/L}=γ_{L/G}, then θ=180° is achieved. This situation corresponds to a type of widely observed wetting phenomena in nature as bouncing water drops in air or on a thin film of air with a “disappeared” S/L interface. As used herein, the subscript “S” refers to solid, the subscript “L” refers to liquid, the subscript “G” refers to gas, the subscript “W” refers to water or an aqueous liquid, and the subscript “O” refers to oil or an oil-based solution or suspension.

Rewriting the subscript of each parameter in Young's equation to meet the undermedia condition gives γ_{S/Lcp}=γ_{S/Ldp}+γ_{Ldp/Lcp} cos θ. Setting θ to 180° yields γ_{S/Lcp}=γ_{S/Ldp}+γ_{Ldp/Lcp}. Next, applying the thermodynamic boundary conditions of surface (γ_{S/G}, γ_{Lcp/G}, and γ_{Ldp/G}>0) and S/L, L/L interfaces (γ_{S/Lcp}<γ_{S/G}+γ_{Lcp/G}, γ_{S/Ldp}<γ_{S/G}+γ_{Ldp/G}, and γ_{Ldp/Lcp}<γ_{Ldp/G}+γ_{Lcp/G}, it is observed that the relationship between γ_{S/Ldp} and γ_{S/Lcp}+γ_{Ldp/Lcp} can be either “>”, “=” or “<”. In other words, γ_{S/Lcp}+γ_{Ldp/Lcp}≤γ_{S/Ldp} becomes obtainable in thermodynamics when the gas phase in Young's equation is replaced by a second liquid phase predicting that a solid capable of perfectly repelling liquid in liquid (e.g., aqueous selected mixture within oil) with long-term stability can exist. Interfacial tensions between each of the selected mixture, the oil, and the surface (e.g., selected mixture-oil interfacial tension, mixture-surface interfacial tension, and oil-surface interfacial tension), can be measured and/or estimated by methods known to those having ordinary skill in the art.

One example of a suitable method for estimating interfacial tensions between a liquid and a solid is measuring contact angles (θ) in air using a goniometer and estimating the interfacial tensions using Young's equation. From Young's equation γ_{S/G}=γ_{S/L}+γ_{L/G} cos θ, γ_{S/L}=γ_{S/G}−γ_{L/G} cos θ can be easily derived. It is worth noting that the range of value of θ in Young's equation is 0°<θ<180°. When θ is measured as 0°, γ_{S/L} calculated as γ_{S/G}−γ_{L/G} represents the possible maximum value, γ_{S/L} Max. In that case, the true γ_{S/L} can be either equal to or smaller than γ_{S/L Max}. If, for example,

$${γ}_{\frac{Surface}{Selected Mixture}} \geq {γ}_{Surface / Oil} + {γ}_{Selected Mixture / Oil} ,$$

Surface/Selected Mixture interface will disappear, which represents underoil perfect water repellency. In other words, underoil perfect water repellency may be achieved when the selected mixture-surface interfacial tension is greater than or equal to the sum of the selected mixture-oil interfacial tension and the oil-surface interfacial tension, thereby giving rise to perfect liquid repellency between the selected mixture and the surface.

Underoil perfect water repellency utilized by the methods, systems, and kits described herein enables low sample loss by preventing biofouling of the substrate surface. Minimizing sample losses to biofouling in turn enables cell separation, isolation, and purification methods on very low sample volumes (e.g., under 1 mL), which have been historically considered too small to be usable. Further, immersing the aqueous selected mixture under oil prevents changes in concentration due to water evaporation.

The term “aqueous sample” refers to a sample containing any liquid composed of water in major and its solutes and/or dispersed phases, including at least one cell described elsewhere herein. Examples of aqueous samples a may include blood, homogenized tissues or organs in aqueous solutions, or mucosal secretions. Aqueous samples may be treated for sample preservation (e.g., treated with anticoagulation agents) or case of sample handling (e.g., diluted, concentrated) in advance of using the methods, systems, and kits disclosed herein. In some cases, the volume of the aqueous sample may be less than 1 mL. In some cases, the volume of the aqueous sample used in the methods, systems, and kits disclosed herein may be less than about 500 μL, less than about 400 μL, less than about 300 μL, less than about 200 μL, less than about 150 μL, less than about 100 μL, less than about 90 μL, less than about 80 μL, less than about 70 μL, less than about 60 μL, less than about 50 μL, less than about 40 μL, less than about 30 μL, less than about 20 μL, less than about 10 μL, less than about 9.0 μL, less than about 8.0 μL, less than about 7.0 μL, less than about 6.0 μL, less than about 5.0 μL, less than about 4.0 μL, less than about 3.0 μL, less than about 2.0 μL, or less than about 1.0 μL.

The term “cell” or “cells” as used herein, refers to the structural, functional, and biological units of living beings. As used herein, “cells” include structural, functional, and biological units which make up tissues, organs, organ systems, or organisms. Examples of ‘cells’ include stem cells, supporting cells, secretory cells exocrine, secretory cells endocrine, secretory cells matrix, phagocytic cells, killing cells, ion-transporting cells, antigen-presenting cells, contracting cells, endothelial cells, sensory cells, and nerve cells. “Cells,” for example, may include monocytes, lymphocytes, neutrophils, cosinophils, basophils, macrophages, erythrocytes. Other examples of “cells” may include parts of cells (e.g., cell fractions), such as platelets, lysed cells, subcellular components, organelles, or biological macromolecules (e.g., proteins, protein complexes, chromatin, nucleic acids, carbohydrates, or lipids). “Cell” may also refer to bacteria, viruses, or single-cell organisms. Aqueous samples may contain a mixture of cells. For example, the aqueous sample may contain a first cell, a second cell, a third cell, etc. In some embodiments, the first cell may be distinguishable over other cells present in the aqueous sample by one or more primary binding members, such as a first primary binding member. Similarly, a second cell may be distinguishable over other cells present in the aqueous sample by a second primary binding member, wherein the first primary binding member and second primary binding member arc different primary binding members. In some cases, the primary binding member is an antigen. In some cases, the primary binding member is a surface antigen (i.e., an antigen on the outward-facing surface of the cell). For example, antigens may include CD19, CD14, CD56, glycophorin A to distinguish B cells, monocytes, NK cells and red blood cells.

The methods, systems, and kits further include a secondary binding member reactive to at least one primary binding member of at least one cell. In some cases, the secondary binding member is an antibody. In some cases, multiple secondary binding members may be used to target a primary binding member or primary binding members of one or more than one cell present in the aqueous sample. Secondary binding members may be selected based upon their selectivity for a primary binding member of a target cell to be isolated, purified, or separated (e.g., a positive selection). In other cases, secondary binding members may be selected based on its non-reactivity with a target cell's primary binding member, and for its reactivity with one or more cells intended for removal (e.g., a negative selection). The reaction chemistry between the primary binding member and the secondary binding member may include the reaction chemistry of antibodies and antigens, electrostatic In some cases, a mixture of secondary binding members (e.g., a secondary binding member cocktail, an antibody cocktail) may be used to target multiple cells present in the aqueous sample. The secondary binding members further include a binding site reactive to the magnetic particle's binding site. In some cases, the binding site may employ streptavidin-biotin chemistry.

The methods, systems, and kits disclosed herein include a magnetic particle. Magnetic particles may include single particles or aggregates of several particles (i.e., particulates) which may be manipulated with a magnetic field. The magnetic particles may be nanoparticles or micro-scale particles (e.g., the magnetic particles may have at least one dimension no less than 0.5 nm and no greater than 1000 μm). They may include a magnetic material (e.g., iron, nickel, cobalt). The magnetic particles disclosed herein include a particle binding site reactive to one or more secondary binding members. In some cases, particle binding sites may employ streptavidin-biotin chemistry.

The methods, systems, and kits disclosed herein may include one or more components or reagents (e.g., a mixture of secondary binding members as an antibody cocktail, magnetic particles) of cell separation kits already known in the art. Cell separation kits may include negative selection kits and positive selection kits. For example, T cell negative isolation kits, neutrophil negative isolation kits, NK cell negative isolation kits, monocyte isolation kits. Exemplary cell separation kits include EasySep™ cell separation kits, such as EasySep™ Human cell separation kits, EasySep™ mouse and rat cell separation kits, and EasySep™ for other species and cell types. Other exemplary cell separation kits include EasySep™ Direct kits, such as EasySep™ Direct T cell isolation kit. In some cases, the cell separation kit may include a mixture of secondary binding members (e.g., an antibody cocktail) targeting lineage markers of cells which are not the cell(s) of interest (i.e., a negative selection). For example, to isolate T cells from an aqueous sample including whole blood, a secondary binding member cocktail may contain secondary binding members which are reactive to be CD19, CD14, CD56, glycophorin A to remove B cells, monocytes, NK cells, and red blood cells.

The methods, systems, and kits include exposing the aqueous sample, including a first cell, to the first secondary binding member and to the magnetic particle may form a selected mixture. The selected mixture includes a first magnetically tagged cell with the first secondary binding member bound to the particle binding site of the magnetic particle, and the first secondary binding member bound to the first primary binding member of the first cell. Specific reaction conditions and durations of exposure may be described elsewhere in the art, including in exemplary cell separation kits such as the aforementioned EasySep™ cell separation kits. In some cases, it may be necessary to scale down one or more components or reagents (e.g., a mixture of secondary binding members as a secondary binding member cocktail, magnetic particles) of cell separation kits already known in the art to accommodate the volume of the aqueous sample. In some cases, the volume of the selected mixture may be less than 1 mL. In some cases, the volume of the selected mixture may be less than about 500 μL, less than about 400 μL, less than about 300 μL, less than about 200 μL, less than about 150 μL, less than about 100 μL, less than about 90 μL, less than about 80 μL, less than about 70 μL, less than about 60 μL, less than about 50 μL, less than about 40 μL, less than about 30 μL, less than about 20 μL, less than about 10 μL, less than about 9.0 μL, less than about 8.0 μL, less than about 7.0 μL, less than about 6.0 μL, less than about 5.0 μL, less than about 4.0 μL, less than about 3.0 μL, less than about 2.0 μL, or less than about 1.0 μL.

In another aspect, the methods, systems, and kits described herein include placing the selected mixture on a surface of a substrate. The substrate may include a substantially flat surface or a curved surface, such as a dimple, well, or cup. For example, the substrate may be a centrifuge tube, a microcentrifuge tube, a cell culturing plate, a vial, or an array of wells (e.g., a well plate or a microwell plate). The surface of the substrate may be substantially flat, conical, round, or V-shaped. In some cases, the substrate may have a curved surface which is matched to the curvature of the droplet of selected mixture such that the droplet of the selected mixture may be centered on the lowest point of the curved surface. In some cases, the curvature of the surface may confine the droplet of the selected mixture to a predetermined location on the substrate for case of liquid handling by, for example, a liquid handling robot. By way of example, for a selected mixture having a volume of about 8.0 μL, a 0.6 mL microcentrifuge tube (e.g., an 0.6-mL Eppendorf® tube) having a bottom diameter of 3.2 mm may be used. In another example, for a selected mixture having a volume of about 70 μL, a 5-mL microcentrifuge tube (e.g., a 5-mL Axygen® tube) having a bottom diameter of 6.4 mm may be used. The substrate may include polypropylene, polycarbonate, polystyrene, polyethylene, polytetrafluoroethylene, polydimethylsiloxane, glass, and any combinations thereof.

The surface of the substrate includes an oil (i.e., a liquid immiscible in water and/or an aqueous liquid). The oil may thinly coat the surface (e.g., a molecularly thin layer of oil) or the surface may be immersed in oil so that the selected mixture, when place on the surface of the substrate, is also completely immersed in the oil. It may be desirable to use a volume of oil sufficient to completely immerse the selected mixture and completely immerse volumes of subsequently separated magnetically tagged cells attracted to a magnetic field. In some cases, the volume of the selected mixture is less than half of the volume of the oil on the surface (i.e., the volume ratio of the oil to the selected mixture is greater than 2:1). By way of example, for a selected mixture having a volume of about 8.0 μL and where the substrate is a 0.6 mL microcentrifuge tube, the oil volume residing in the microcentrifuge tube may be about 20 μL (e.g., the volume ratio of the oil to the selected mixture is about 2.5:1). Smaller volume ratios of oil to selected mixture may be possible. The oil may include silicone oil, fluorinated oil, mineral oil, and any combinations thereof.

In another aspect, the methods, systems, and kits described herein include exposing the selected mixture to a magnetic field and magnetically attracting the magnetically tagged cell to the magnetic field. The magnetic field may be produced by a permanent magnet (e.g., a rare-earth magnet, a ceramic magnet, an alnico magnet, and combinations thereof), a temporary magnet, an electromagnet, and any combinations thereof. In some cases, the magnetic field may be produced by a rare-earth magnet installed in the tip of pipette. In some cases, the magnets are brought close to the selected mixture. In other cases, the magnets contact the selected mixture. The magnetic field strength and shape may be tailored to the volume of the selected mixture so that the entire volume of selected mixture is exposed to a sufficient magnetic field to attract the magnetically tagged cells to the magnet. For example, for a selected mixture having a volume of about 8.0 μL, a rare-earth magnet having a pull force of about 0.15 lbs. and dimensions of 1/16″ dia.×⅛″ thickness may be used. In another example, for a selected mixture having a volume of about 70 μL, a rare-earth magnet having a pull force of about 2.28 lbs. and dimensions of 3/16″ dia.× 3/16″ thickness may be used. The selected mixture may be exposed to the magnetic field for an exposure time. In some cases, the duration of the exposure time is no greater than 5 minutes, is no greater than 4 minutes, is no greater than 3 minutes, is no greater than 2 minutes, is no greater than 1 minute, is no greater than 45 seconds, is no greater than 30 seconds, no greater than 20 seconds, no greater than 15 seconds, or no greater than 10 seconds.

The exposure of the magnetically tagged cells to a magnetic field separates the magnetically tagged cells from the aqueous sample to produce a separated aqueous sample. The separated aqueous sample may be substantially free from the magnetically tagged cell. As used herein, “substantially free from” refers to a frequency of magnetically tagged cells in the separated aqueous sample of less than 20%, less than 15%, less than 10%, less than 5%, less than 2.0%, less than 1.0%, or less than 0.5% based on the total cell count. Referring to FIG. 1, the methods described herein may be performed in a single step (100). In some cases, the single step is a multiplexed step for either positively or negatively selecting a target cell for isolation, separation, or purification. In some cases, the single step may utilize a mixture of multiple secondary binding members (first, second, third, etc. secondary binding members) targeting multiple primary binding members (first, second, third, etc. primary binding members) correlating to multiple cell types (first, second, thirds, etc. cells) present in the aqueous sample. This simultaneous, multiplexed formation of first, second, third, etc. magnetically tagged cells in the selected mixture may be separated, isolated, or purified by exposing the selected mixture to a magnetic field to magnetically attract the first, second, third magnetically tagged cells to the magnet simultaneously. This single step may produce a separated aqueous sample substantially free from the first, second, third, etc. magnetically tagged cells.

Still referring to FIG. 1, the methods described herein may be performed in two or more serial steps (200). For example, a first secondary binding member targeting a first primary binding member correlating to a first cell type present in the aqueous sample to produce a first magnetically tagged cell in the first selected mixture. The first magnetically tagged cell may be separated, isolated, or purified by exposing the first selected mixture to a magnetic field to magnetically attract the first magnetically tagged cell to the magnet to produce a first separated aqueous sample substantially free from the first magnetically cells. In some cases, the first separated aqueous sample may be removed from being immersed under the oil using a pipette and the following second steps (9-13) may be performed away from or off of the substrate including an oil. In other cases, the first separate aqueous sample may remain immersed under the oil and/or on the surface of the substrate for the second steps (9-13).

Still referring to FIG. 1, in a second step, the first separated aqueous sample (9) may be exposed to a second magnetic particle (10), a second secondary binding member (11) targeting a second primary binding member correlating to a second cell type (e.g., a second cell) present in the aqueous sample to produce a second magnetically tagged cell (12) in a second selected mixture. Optionally, if the first separate aqueous sample was removed from under the oil and the second selected mixture was produced above or outside of the oil, the second selected mixture may be placed on a surface of a substrate (13), the surface including an oil, exposing the selected mixture to a magnetic field (14) and magnetically attracting the first magnetically tagged cell to the magnetic field, and separating (15) the first magnetically tagged cell (16) from the aqueous sample to produce a second separated aqueous sample (17).

The second magnetically tagged cell may be separated, isolated, or purified by exposing the second selected mixture to a magnetic field to magnetically attract the second magnetically tagged cell to the magnet to produce a second separated aqueous sample substantially free from both the first magnetically tagged cells and second magnetically tagged cells. Subsequent serial steps may be similarly performed to produce a third separated aqueous sample substantially free from both the first magnetically tagged cells, second magnetically tagged cells, and third magnetically tagged cells. In other cases, the method disclosed herein may be performed with initial multiplexed step followed by a single positive selection. For example, a human dendritic cell isolation kit (e.g., CD1c (BDCA-1)⁺ Dendritic Cell Isolation Kit, human by Miltenyi Biotec) may initially remove CD19+ B cells and CD14+ monocytes in a first step. In a subsequent positive selection, CD1c dendritic cells may be selected.

Referring to FIG. 2, a cell separation system is disclosed herein. The system includes a surface of a substrate (20) and the surface includes at least one oil (21). The system further includes a source of a magnetic field (26). In some cases, the oil has an oil volume to create an oil fill line (211) sufficient to keep the selected mixture and subsequently separated volumes of magnetically tagged cells attracted to a magnetic field. The system further includes a secondary binding member (3) having a primary binding member binding site reactive to a primary binding member and a binding site reactive to a magnetic particle binding site. The system further includes a magnetic particle (2) comprising a magnetic particle binding site corresponding to the secondary binding member. The system includes a set of instructions executed by a liquid handling machine (24). The liquid handling machine may include a manually operated pipet or a computer-controlled liquid handling robot. The liquid handling machine may also be a human being with a pipette. The instructions include providing an aqueous sample (1) and exposing the aqueous sample (22), including a cell having a primary binding member to the magnetic particle and to the secondary binding member to form a selected mixture (23). The selected mixture includes a magnetically tagged cell including the secondary binding member bound to the magnetic particle binding site of the magnetic particle, and the secondary binding member bound to the primary binding member of the cell. As seen in FIG. 2A-2B, the instructions further include placing the selected mixture (23) on a surface of a substrate (20), the surface having an oil (21). The instruction further includes exposing the selected mixture to a magnetic field (25) and magnetically attracting the magnetically tagged cell to the magnetic field. As shown in FIG. 2D, the instructions also include separating the magnetically tagged cell from the aqueous sample (250) to produce a separated aqueous sample substantially free of the magnetically tagged cell (28). In some cases, the separated magnetically tagged cells may be physically partitioned into a separate droplet (27) which is able to be removed from the separated aqueous sample.

Also disclosed herein are kits for cell separation. The kits include a secondary binding member having a primary binding member binding site reactive to a primary binding member and a binding site reactive to a magnetic particle binding site, a magnetic particle having a magnetic particle binding site corresponding to the secondary binding member, a surface of a substrate, the surface comprising an oil, and a source of a magnetic field. Suitable secondary binding members, magnetic particles, substrates, oils, and sources of magnetic fields are described above.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are provided in order to demonstrate and further illustrate certain embodiments and aspects of the present technology and are not to be construed as limiting the scope of the technology. The statements provided in the Examples are presented without being bound by theory.

### Inventive Method of Cell Separation

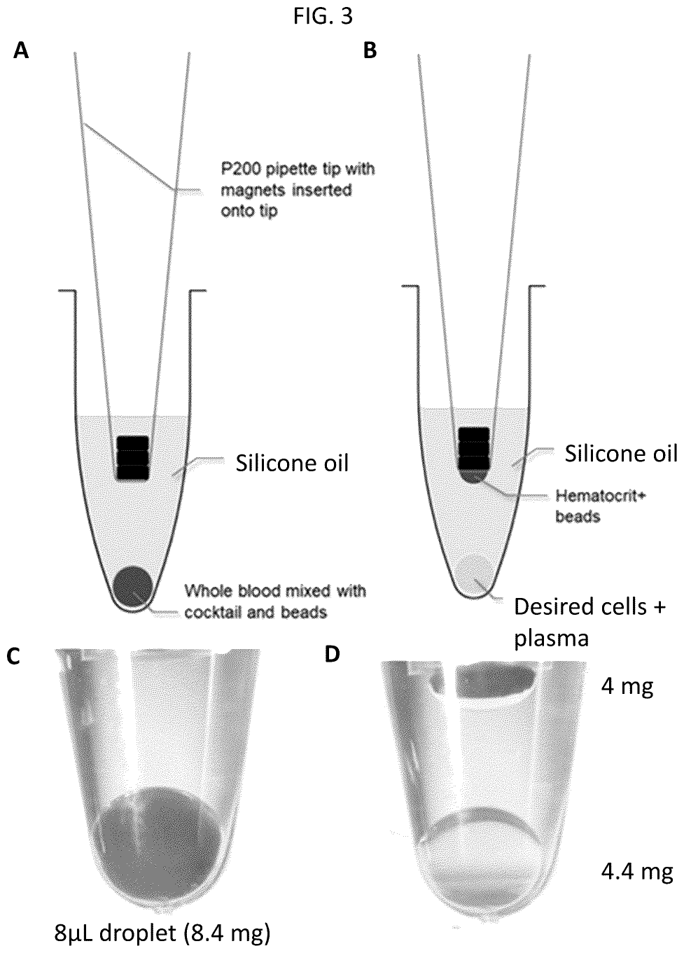

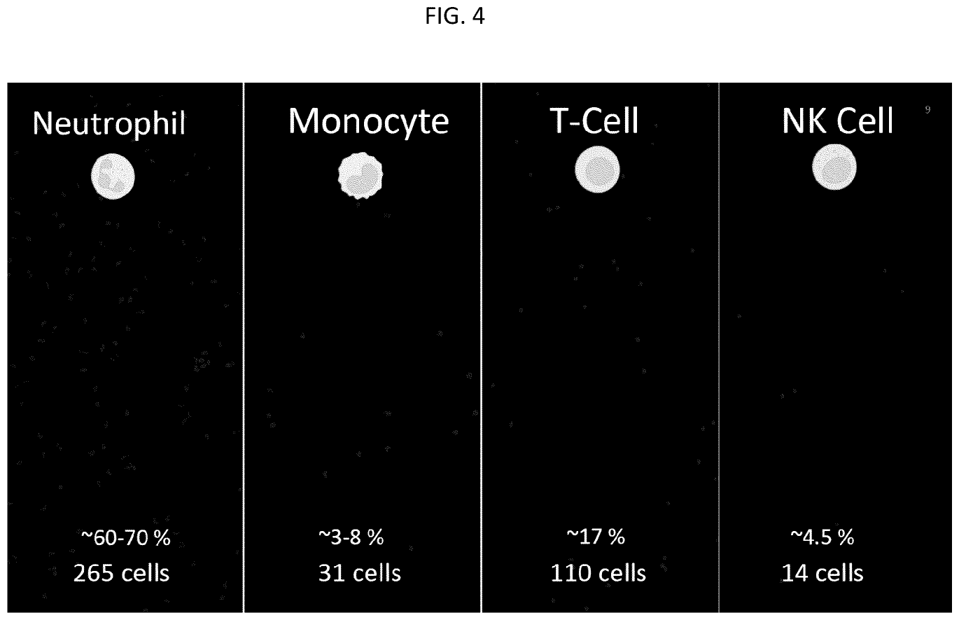

Peripheral blood mononuclear cells, specifically neutrophils, monocytes, T cells, and natural killer (NK) cells, were isolated from microvolume blood samples (8 μl) obtained from a single donor using the methods, systems, and kits disclosed herein. The blood was drawn by standard finger prick and immediately treated with acid citrate dextrose (ACD) as an anticoagulant at a 1:10 ratio. The isolation of different cell types was performed using negative isolation kits (EasySep™ Direct Human T Cell Isolation Kit Cat #19661, EasySep™ Direct Human NK Cell Isolation Kit Cat #19665, EasySep™ Direct Human Neutrophil Isolation Kit Cat #19666, and EasySep™ Direct Human Monocyte Isolation Kit Cat #19669) with reagents proportionally scaled down based on the manufacturer's recommendations for 1.5 ml blood samples. Each blood droplet received the addition of the cell-specific antibody cocktail and the magnetic beads, followed by the addition of a volume of PBS equal to the initial blood volume. The mixture was then transferred into 20 μl of silicone oil-filled Eppendorf tube followed with a five-minute incubation period at room temperature (FIG. 2A-2B). The non-target cells were removed with a magnet for 1 min (FIG. 2C and 3a2), leaving the purified cells to be collected by pipetting.

For 8 μl isolation, a standard 0.6 ml Eppendorf tube with a bottom diameter of 3.2 mm is used. For a 70 μl isolation, a standard 5 ml Axygen MCT-500-C tube with a bottom diameter of 6.4 mm is used. his can be modified based on the size of the droplet and can be made compatible with other tubes or scaled up by using round bottom plates.

### Purity of Separated Cells

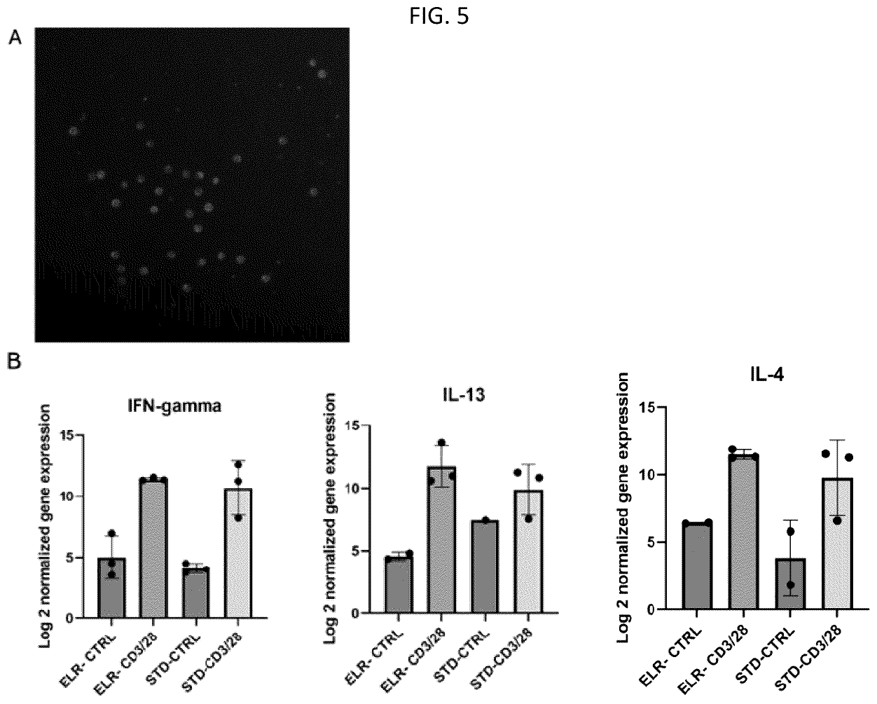

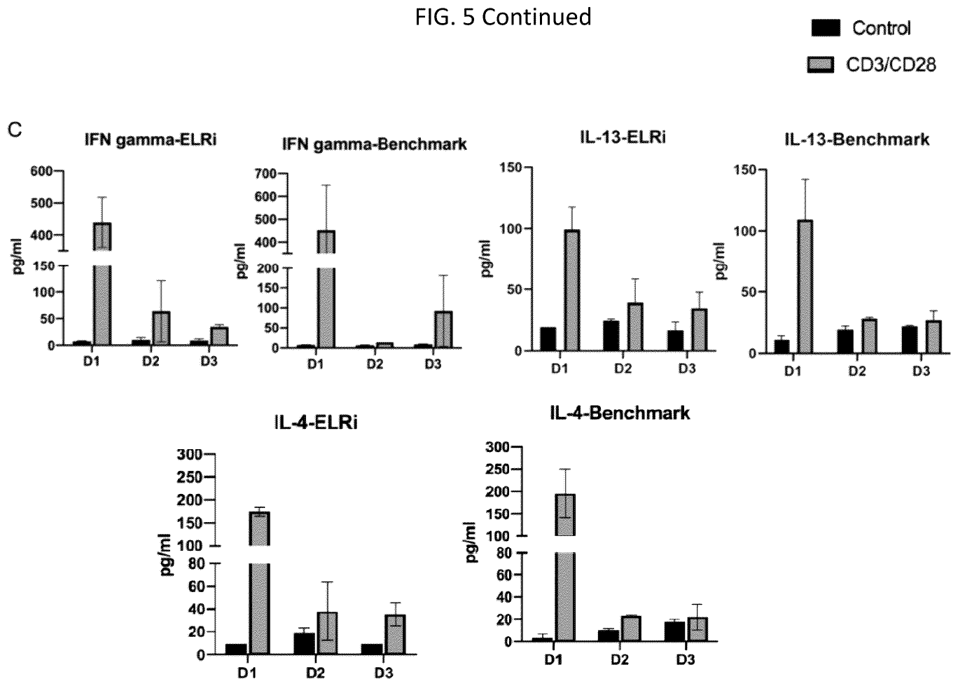

Subsequently, the cells were stained with Hoechst dye for both quantification and morphological assessment. The stained cells were then loaded into a standard 96-well plate with media containing 10 μg/ml Hoechst and allowed to adhere for 30 minutes. Analysis of the stained cells revealed that the frequency of isolated cells, as shown in FIG. 4, consistent with the known frequencies of these cells in whole blood, indicating that the methods disclosed herein provide a uniform isolation efficiency across different cell types. Isolated T cells were then specifically stained with Hoechst at 10 μg/ml to visualize nuclei and with an anti-CD3 antibody (Alexa Fluor® 594 anti-human, Biolegend, Cat #300446) at 10 μg/ml for 30 min on ice to confirm T cell purity. Cells were washed three times with PBS prior to imaging on a fluorescent microscope. Cells were quantified based on the presence of CD3 stained nuclei compared to non CD3 positive nuclei resulting in an approximate purity of 95%, as depicted in FIG. 5A.

### Functionality of Separated T Cells

10 ml of blood was acquired by venipuncture in an ACD vacutainer. T-Cells were isolated from 30 μl of blood using the methods disclosed herein. 1.5 mL of blood was used to isolate T-Cells using the EasySep™ Direct Human T Cell Isolation Kit Cat #19661 according to manufactures instructions. Post-isolation, T cells were either stimulated with ImmunoCult™ Human CD3/CD28 T Cell Activator (Stemcell, Cat #10971) according to the manufacturers recommendations or left unstimulated as a control. Gene expression analyses for T-helper type 1 (Th1) (IFN gamma) and type 2 (Th2) associated (IL-4, IL-13) genes were performed using quantitative PCR (qPCR). Briefly, RNA was extracted using the RNeasy kit (Qiagen, Cat #74104) per the manufacturer's recommendations. RNA was converted to cDNA using High-Capacity RNA-to-cDNA™ Kit (Thermo, Cat #4387406) and then pre-amped using (SsoAdvanced PreAmp Kit, Biorad, Cat #1725160). Taqman primers were purchased from Thermo, and qPCR was run on a Roche Light cycler 480. Gene expression data were compared and normalized against three house-keeping genes. These genes were consistent across multiple donors and highly similar between the methods disclosed herein and standard isolation methods, indicating equivalent functionality between T cells isolated by the methods disclosed herein and those isolated using standard volume (STD) methods, as shown in FIG. 5B.

Supernatant from the T-cells after two days of incubation from the above experiment was analyzed using Thermo Procartaplex custom multiplex bead-based ELISA according to manufacturer's instructions. This assay quantified cytokines that are markers for Th1 (IFN-gamma) and Th2 responses (IL-4, IL-13). The ELISA results corroborated the qPCR findings, with similar cytokine secretion patterns observed in both the methods disclosed herein and standard preparations across three different donors, as demonstrated in FIG. 5C.

What is claimed is:

1. A method for cell separation, comprising:
providing an aqueous sample comprising a first cell, the first cell comprising a first primary binding member;
providing a magnetic particle comprising a particle binding site;
providing a first secondary binding member comprising a first primary binding member binding site reactive to the first primary binding member and a binding site reactive to the particle binding site;
exposing the aqueous sample to the first secondary binding member and to the magnetic particle to form a selected mixture comprising:
a first magnetically tagged cell comprising:
the first secondary binding member bound to the particle binding site of the magnetic particle, and
the first secondary binding member bound to the first primary binding member of the first cell;
placing the selected mixture on a surface of a substrate, the surface comprising an oil;
exposing the selected mixture to a magnetic field and magnetically attracting the first magnetically tagged cell to the magnetic field; and
separating the first magnetically tagged cell from the aqueous sample to produce a separated aqueous sample.

2. The method of claim 1, wherein providing an aqueous sample comprising a first cell, the first cell comprising a first primary binding member, further comprises:
providing an aqueous sample comprising a first cell, the first cell comprising a first primary binding member,
wherein the first primary binding member is an antigen; and
wherein providing a first secondary binding member comprising a first primary binding member binding site reactive to the first primary binding member and a binding site reactive to the particle binding site, further comprises:
providing a first secondary binding member comprising a first primary binding member binding site reactive to the first primary binding member and a binding site reactive to the particle binding site,
wherein the secondary binding member is an antibody.

3. The method of claim 1, wherein placing the selected mixture on a surface of a substrate, the surface comprising an oil, further comprises:
the selected mixture and the oil having a known selected mixture-oil interfacial tension;
the selected mixture and the surface having a known selected mixture-surface interfacial tension;
the oil and the surface having a known oil-surface interfacial tension; and
wherein the selected mixture-surface interfacial tension is greater than or equal to the sum of the selected mixture-oil interfacial tension and the oil-surface interfacial tension, thereby giving rise to perfect liquid repellency between the selected mixture and the surface.

4. The method of claim 1, wherein separating the first magnetically tagged cell from the aqueous sample to produce a separated aqueous sample further comprises:
separating the first magnetically tagged cell from the aqueous sample to produce a separated aqueous sample,
wherein the separated aqueous sample is substantially free of the first magnetically tagged cell.

5. The method of claim 1, further comprising:
wherein providing an aqueous sample comprising a first cell, the first cell comprising a first primary binding member further comprises:
providing an aqueous sample comprising a second cell, the second cell comprising a second primary binding member;
wherein providing a first secondary binding member comprising a first primary binding member binding site reactive to the first primary binding member and a binding site reactive to the particle binding site further comprises:
providing a second secondary binding member comprising a second primary binding member binding site reactive to the second primary binding member and a binding site reactive to the particle binding site;
wherein exposing the aqueous sample to the first secondary binding member and to the magnetic particle to form a selected mixture further comprises:
exposing the aqueous sample to the magnetic particle and to the second secondary binding member to form a selected mixture comprising:
a second magnetically tagged cell comprising:
the second secondary binding member bound to the particle binding site of the magnetic particle, and
the second secondary binding member bound to the second primary binding member of the second cell;
wherein exposing the selected mixture to a magnetic field and magnetically attracting the first magnetically tagged cell to the magnetic field further comprises:
exposing the selected mixture to a magnetic field and magnetically attracting the second magnetically tagged cell to the magnetic field; and
wherein separating the first magnetically tagged cell from the aqueous sample to produce a separated aqueous sample further comprises:
separating the second magnetically tagged cell from the aqueous sample to produce a separated aqueous sample.

6. The method of claim 5, wherein separating the second magnetically tagged cell from the aqueous sample to produce a separated aqueous sample further comprises:
wherein separating the second magnetically tagged cell from the aqueous sample to produce a separated aqueous sample,
wherein the separated aqueous sample is substantially free of the second magnetically tagged cell.

7. The method of claim 1, wherein placing the selected mixture on a surface of a substrate further comprises:
placing the selected mixture on the surface of the substrate,
wherein the surface of the substrate is a well and the oil at least coats the surface.

8. The method of claim 1, wherein placing the selected mixture on a surface of a substrate further comprises:
placing the selected mixture on the surface of the substrate,
wherein the selected mixture is immersed the oil.

9. The method of claim 1, wherein placing the selected mixture on a surface of a substrate further comprises:
placing the selected mixture on the surface of the substrate,
wherein the volume ratio of the oil to the selected mixture is greater than 2:1.

10. The method of claim 1, wherein providing an aqueous sample comprising a first cell, the first cell comprising a first primary binding member further comprises:
providing an aqueous sample comprising a first cell, the first cell comprising a first primary binding member,
wherein the first primary binding member is a surface antigen.

11. The method of claim 1, wherein exposing the aqueous sample to the magnetic particle and to the first secondary binding member to form a selected mixture further comprises:
exposing the aqueous sample to the magnetic particle and to the first secondary binding member to form a selected mixture,
wherein the selected mixture is less than 1 mL.

12. The method of claim 1, wherein exposing the selected mixture to a magnetic field and magnetically attracting the first magnetically tagged cell to the magnetic field further comprises:
exposing the selected mixture to a magnetic field and magnetically attracting the first magnetically tagged cell to the magnetic field,
wherein the selected mixture is exposed to the magnetic field for an exposure time, and
wherein the exposure time is no greater than 1 minute.

13. The method of claim 1, wherein exposing the aqueous sample to the first secondary binding member and to the magnetic particle to form a selected mixture further comprises:
exposing the aqueous sample to a first secondary binding member and a magnetic particle of a T cell negative isolation kit, neutrophil negative isolation kit, NK cell negative isolation kit, monocyte isolation kit, or any combinations thereof.

14. The method of claim 8, further comprising:
wherein the separated aqueous sample comprises a second cell, the second cell comprising a second primary binding member;
providing a second magnetic particle comprising a second particle binding site;
providing second secondary binding member comprising second primary binding member binding site reactive to the second primary binding member and second binding site reactive to the particle binding site;
exposing the separated aqueous sample to the second magnetic particle and to the second secondary binding member to form a second selected mixture comprising:
a second magnetically tagged cell comprising:
the second secondary binding member bound to the particle binding site of the second magnetic particle, and
the second secondary binding member bound to the second primary binding member of the second cell; and
exposing the second selected mixture to a magnetic field and magnetically attracting the second magnetically tagged cell to the magnetic field; and
separating the second magnetically tagged cell from the aqueous sample to produce a second separated aqueous sample substantially free of the second magnetically tagged cell.

15. A system for cell separation, comprising:
a secondary binding member comprising a primary binding member binding site reactive to a primary binding member and a binding site reactive to a magnetic particle binding site;
a magnetic particle comprising a magnetic particle binding site corresponding to the secondary binding member;
a surface of a substrate, the surface comprising an oil;
a source of a magnetic field; and
a set of instructions executed by a liquid handling machine comprising:
exposing an aqueous sample, comprising a cell comprising a primary binding member to the magnetic particle and to the secondary binding member to form a selected mixture comprising:
a magnetically tagged cell comprising:
the secondary binding member bound to the magnetic particle binding site of the magnetic particle, and
the secondary binding member bound to the primary binding member of the cell;
placing the selected mixture on a surface of a substrate, the surface comprising an oil;
exposing the selected mixture to a magnetic field and magnetically attracting the magnetically tagged cell to the magnetic field; and
separating the magnetically tagged cell from the aqueous sample to produce a separated aqueous sample substantially free of the magnetically tagged cell.

16. A kit for cell separation, comprising:
a secondary binding member comprising a primary binding member binding site reactive to a primary binding member and a binding site reactive to a magnetic particle binding site;
a magnetic particle comprising a magnetic particle binding site corresponding to the secondary binding member;
a surface of a substrate, the surface comprising an oil; and
a source of a magnetic field.

17. The kit of claim 16, wherein the surface of a substrate is selected from the group consisting of polypropylene, polycarbonate, polystyrene, polyethylene, polytetrafluoroethylene, polydimethylsiloxane, glass, and any combinations thereof.

18. The kit of claim 16, wherein the substrate is selected from the group consisting of a centrifuge tube, a microcentrifuge tube, a well plate, and a cell culturing plate.

19. The kit of claim 16, wherein the oil is selected from the group consisting of silicone oil, fluorinated oil, mineral oil, and any combinations thereof.

20. The kit of claim 16, wherein the source of a magnetic field is selected from the group consisting of a permanent magnet, a temporary magnet, an electromagnet, and any combinations thereof.
